(19) United States
(12) Patent Application Publication
(10) Pub. No.: US 2025/0186070 A1
(43) Pub. Date: Jun. 12, 2025

(54) CLOT ENGAGEMENT DETECTION IN THROMBECTOMY SYSTEMS

(71) Applicant: Walk Vascular, LLC, Irvine, CA (US)

(72) Inventors: Seung Hae Clare CHUNG, Los Altos, CA (US); Ted SU, Sunnyvale, CA (US); Matthew J. GILLICK, Murrieta, CA (US); Caroline KIM, Signal Hill, CA (US); Dustin M. COSSIO, Brea, CA (US); Stephen C. OLSON, Temecula, CA (US); Nianjiong BEI, Palo Alto, CA (US); Adam K. HOOPAI, Highland Park, IL (US)

(21) Appl. No.: 18/968,896

(22) Filed: Dec. 4, 2024

Related U.S. Application Data
(60) Provisional application No. 63/607,461, filed on Dec. 7, 2023.

(51) Int. Cl. A61B 17/22; A61B 17/00; A61B 90/00

(52) U.S. Cl. CPC A61B 17/22; A61B 17/00234; A61B 90/06; A61B 2017/00039; A61B 2017/00057; A61B 2017/00238; A61B 2017/22079; A61B 2090/064; A61B 2217/005; A61B 2217/007

(57) ABSTRACT

A thrombectomy system includes (i) an aspiration catheter configured for advancement through vasculature of a subject to facilitate clot removal from the vasculature of the subject; and (ii) a clot detection component. The clot detection component can comprise an emitter and a detector, wherein the detector is configured to detect emissions of the emitter to indicate whether a propagation path between the emitter and the detector is occluded to determine the presence of a clot. The clot detection component can comprise a clot detection probe configured to emit a signal and detect a reflected signal that is reflected off of a vessel lumen to determine the presence of a clot. The clot detection component can comprise a clot detection probe implemented as a force or pressure sensor to determine the presence of a clot.

### CROSS-REFERENCE TO RELATED APPLICATION(S)

This application claims priority to U.S. Provisional Patent Application No. 63/607,461, filed on Dec. 7, 2023, and entitled “CLOT ENGAGEMENT DETECTION IN THROMBECTOMY DEVICES”, the entirety of which is incorporated herein by reference for all purposes.

### BACKGROUND

### Technical Field

The present disclosure pertains generally to medical devices and methods of their use. More particularly, the present invention pertains to aspiration and thrombectomy devices and methods of use thereof.

### Description of the Related Technologies

Blood clots (thrombi) can form in various parts of the body and can pose a serious health risk. For instance, blood clots can block blood flow and/or lead to tissue damage, organ dysfunction, or life-threatening conditions like stroke or heart attack. Thrombectomy is a medical procedure used to remove blood clots from blood vessels to restore blood flow and prevent further complications.

Various types of catheter-based thrombectomy devices have been developed to aid in the removal of thrombotic material. Such devices are typically inserted into the affected blood vessel through a small incision or artery access point. Catheter-based thrombectomy devices include mechanical thrombectomy devices, rheolytic thrombectomy devices, and others (e.g., ultrasound-assisted devices).

Mechanical thrombectomy devices can implement various types of mechanical components to engage with and remove thrombotic material. For instance, stent retrievers and clot retriever baskets are designed for navigation through vasculature to the site of a clot, deployment at the clot site to cause the stent retriever or clot retriever basket to entrap the clot, and withdrawal through the vasculature to facilitate clot removal. As another example, suction-based thrombectomy devices use negative pressure to aspirate clots from blood vessels (e.g., via a catheter with a distal tip to be placed near the clot prior to activation to draw the clot into a collection chamber, where it is trapped and removed). As yet another example, rotational thrombectomy devices employ rotational mechanisms to fragment and remove clots (e.g., a rotating wire or catheter tip for creating shear forces that break down clots), allowing the fragments to be cleared by the body or using aspiration or other techniques.

Rheolytic thrombectomy devices employ mechanisms that rely on high-velocity jets to break down and remove thrombotic material. Rheolytic thrombectomy mechanisms may be positioned on catheters (e.g., at or near the distal tip) and can utilize saline solution, or a mixture of saline and the patient's own blood, to create high-velocity jets for direction toward clots to generate shear forces that disrupt the clot's structure. The jetted fluid can cause fragmentation of the clot, and the fragments may then be cleared naturally from the body or by aspiration techniques.

Some thrombectomy devices aspects of suction-based thrombectomy devices and rheolytic thrombectomy devices. For instance, some thrombectomy devices utilize a saline jet positioned at or near a distal tip of an aspiration catheter, allowing for aspiration of clot fragments as the jetted saline macerates the clot (e.g., thrombus and/or soft emboli).

To facilitate clot removal, catheter-based thrombectomy devices typically need to be navigated toward a clot site within patient vasculature. However, users (e.g., healthcare practitioners) often experience difficulty in determining whether a catheter of a thrombectomy device is appropriately positioned to perform clot removal functions. Users often resort to a trial-and-error approach, which can result in excessive patient blood loss (e.g., in systems that implement aspiration), damage to vessel walls, increased treatment times (which can give rise to other patient risks), failed clot removal, and/or other negative outcomes for patients. Furthermore, a lack of visualization of clot engagement can make it difficult for users to determine when clot removal is complete, which can also lead to excessive patient blood loss (e.g., when aspiration processes persist after clot removal), damage to vessel walls (e.g., from repeated clot removal process activations/de-activations), increased treatment times, failed clot removal, and/or other negative outcomes for patients.

The subject matter disclosed herein is not limited to embodiments that operate only in environments such as those described above. Rather, this background is only provided to illustrate one example technology area where some embodiments described herein may be practiced.

### SUMMARY

At least some disclosed embodiments provide a thrombectomy system that includes (i) an aspiration catheter configured for advancement through vasculature of a subject to facilitate clot removal from the vasculature of the subject; and (ii) a clot detection system comprising an emitter and a detector, wherein the emitter and the detector are positioned proximate to a distal end of the aspiration catheter, wherein the detector is configured to detect emissions of the emitter to indicate whether a propagation path between the emitter and the detector is occluded, wherein occlusion of the propagation path between the emitter and the detector indicates a presence of a clot proximate to the distal end of the aspiration catheter.

At least some disclosed embodiments provide a thrombectomy system that includes (i) an aspiration catheter configured for advancement through vasculature of a subject to facilitate clot removal from the vasculature of the subject; and (ii) a clot detection probe extending distally from a distal end of the aspiration catheter, wherein the clot detection probe is configured to emit a signal and to detect a reflected signal that is reflected off of a vessel lumen of the vasculature of the subject to indicate whether a propagation path between the clot detection probe and the vessel lumen is occluded, wherein occlusion of the propagation path between the clot detection probe and the vessel lumen indicates a presence of a clot proximate to the clot detection probe.

At least some disclosed embodiments provide a thrombectomy system that includes (i) an aspiration catheter configured for advancement through vasculature of a subject to facilitate clot removal from the vasculature of the subject; and (ii) a clot detection probe positioned proximate to a distal end of the aspiration catheter, wherein the clot detection probe is configured to detect force or pressure exerted on the clot detection probe, and wherein a detected force or pressure exerted on the clot detection probe that satisfies one or more conditions indicates a presence of a clot proximate to the clot detection probe.

At least some disclosed embodiments provide a thrombectomy system that includes (i) an aspiration catheter configured for advancement through vasculature of a subject to facilitate clot removal from the vasculature of the subject; and (ii) a clot detection system comprising an anode and a cathode, wherein the anode and the cathode are positioned proximate to a distal end of the aspiration catheter, wherein the clot detection system is configured to apply a fixed voltage current at the anode and the cathode, wherein the clot detection system is configured to measure current and/or resistance during application of the fixed voltage current, and wherein the measured current and/or resistance is indicative of a presence of a clot proximate to the distal end of the aspiration catheter.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates a perspective view of an example aspiration catheter.

FIG. 2 illustrates a plan view of example disposable components of a system for aspirating thrombus according to an embodiment of the present disclosure.

FIG. 3 illustrates a sectional view of an example distal end of the aspiration catheter of the system for aspirating thrombus of FIG. 1.

FIG. 4 illustrates a detail view of an example y-connector of the aspiration catheter of the system for aspirating thrombus of FIG. 1.

FIG. 5 illustrates a plan view of example disposable components of a system for aspirating thrombus according to an embodiment of the present disclosure.

FIG. 6 illustrates a perspective view of an example system for aspirating thrombus of FIG. 4

FIGS. 7A and 7B illustrate conceptual representations of a thrombectomy system that includes a clot detection system with an emitter and a detector, according to implementations of the present disclosure.

FIGS. 8A, 8B, 9A, and 9B illustrate conceptual representations of a thrombectomy system that includes a clot detection system with a clot detection probe, according to implementations of the present disclosure.

### DETAILED DESCRIPTION

As indicated hereinabove, many conventional catheter-based thrombectomy devices lack mechanisms to detect whether the catheter is engaged with a clot or not. For example, many aspiration catheters lack a mechanism to determine whether the distal end of the aspiration catheter is engaged with a clot. Users thus often resort to trial and error or other methods to determine when to activate or de-activate aspiration or other clot removal functionalities, which can lead to in excessive patient blood loss (e.g., in systems that implement aspiration), damage to vessel walls, increased treatment times (which can give rise to other patient risks), failed clot removal, and/or other negative outcomes for patients.

The present disclosure pertains to systems, devices, and techniques for detecting clot engagement in thrombectomy systems.

For example, a thrombectomy system may include a clot detection system positioned at or near the distal end of an aspiration catheter. The clot detection system may take on various forms, such as an emitter and detector that are configured to communicate signals to indicate whether thrombotic or other material is positioned between the emitter and the detector. A clot detection system may additionally, or alternatively, be implemented as a clot detection probe configured to emit signals and detected reflected signals to indicate whether thrombotic or other material is positioned between the clot detection probe and a vessel lumen. As another example, a clot detection system may be implemented as a force or pressure sensor configured to indicate whether thrombotic or other material is in contact with the distal end of the aspiration catheter. As yet another example, a clot detection system may be implemented as an anode and cathode configured to apply a fixed voltage current to indicate, based on resistance and/or current measurements, whether a clot is proximate to the distal end of the aspiration catheter.

In some instances, implementing a clot detection system on a thrombectomy device, as described herein, may provide various advantages, such as minimized blood loss while attempting to engage with a clot, minimized blood loss after completing clot aspiration, providing user feedback as to when a clot is detected, confirming whether a clot is present after aspiration, verifying vessel clearance, etc. Such advantages can beneficially facilitate improved patient outcomes.

Although examples discussed herein focus, in at least some respects, on implementing a clot detection system on an aspiration catheter, the techniques and/or components discussed herein may be implemented on other types of catheter-based devices, even outside of the domain of thrombectomy.

### Example Thrombectomy Device

The following discussion relates to an example catheter-based thrombectomy device that implements aspiration aspects and rheolytic aspects and that may comprise or be used to implement at least some disclosed embodiments. As noted above, the principles disclosed herein may be implemented in conjunction with other types of catheter-based thrombectomy systems.

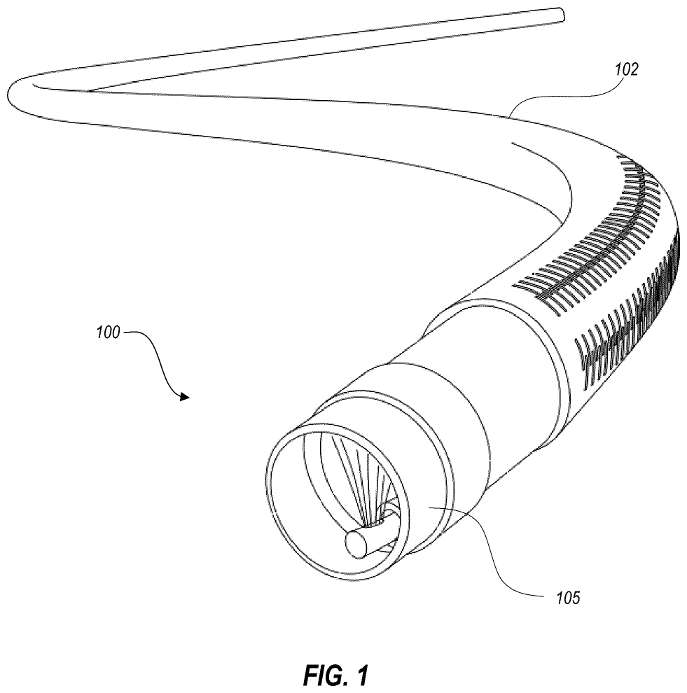

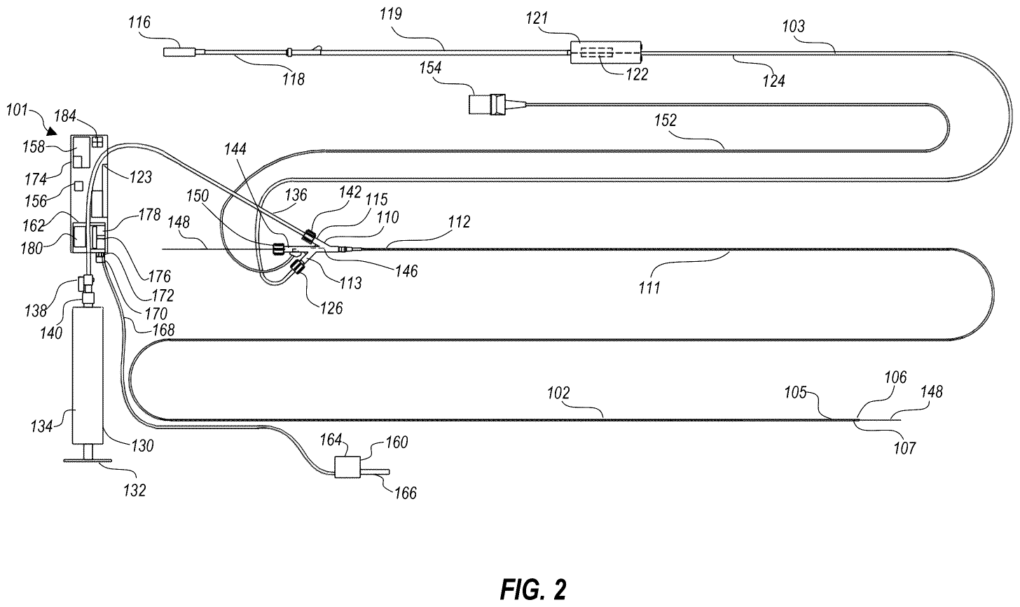

A system 100 for aspirating thrombus is illustrated in FIG. 1, illustrating primarily a distal end 105 of an aspiration catheter 102. FIGS. 2-4 illustrate the system 100 in greater detail. The system 100 for aspirating thrombus includes three major components: a pump 101, an aspiration catheter 102, and a tubing set 103. The aspiration catheter 102 and the tubing set 103 may comprise disposable components. The pump 101 and the pump's associated pump base may comprise reusable components. In some implementations, it is not necessary to sterilize the pump 101, as it may be kept in a non-sterile field or area during use. The aspiration catheter 102 and the tubing set 103 may each be supplied sterile, after sterilization by ethylene oxide gas, electron beam, gamma, or other sterilization methods. The aspiration catheter 102 may be packaged and supplied separately from the tubing set 103, or the aspiration catheter 102 and the tubing set 103 may be packaged together and supplied together. Alternatively, the aspiration catheter 102 and tubing set 103 may be packaged separately, but supplied together (i.e., bundled).

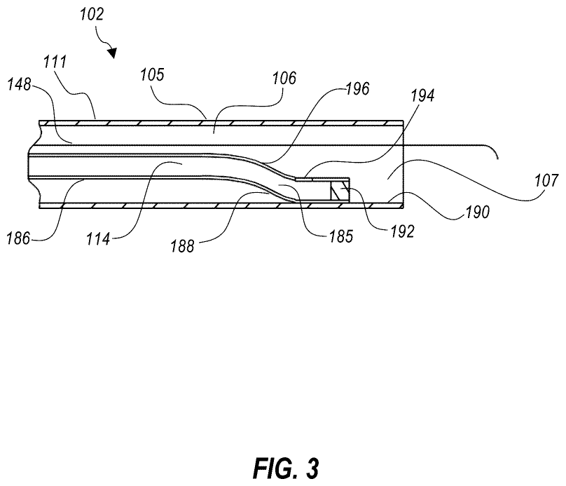

As shown in FIGS. 2-4, the aspiration catheter 102 has a distal end 105 and includes an over-the-wire guidewire lumen/aspiration lumen 106 extending between an open distal end 107, and a proximal end comprising a y-connector 110. The catheter shaft 111 of the aspiration catheter 102 is connected to the y-connector 110 via a protective strain relief 112. In other embodiments, the catheter shaft 111 may be attached to the y-connector 110 with a luer fitting. The y-connector 110 comprises a first female luer 113 which communicates with a catheter supply lumen 114 (FIG. 3), and a second female luer 115 which communicates with the guidewire lumen/aspiration lumen 106.

A spike 116 for coupling to a fluid source (e.g., saline bag, saline bottle) allows fluid to enter through an extension tubing 118 and flow into a supply tube 119. An optional injection port allows injection of materials or removal of air. A cassette 121 having a moveable piston 122 is used in conjunction with a mechanical actuator 123 of the pump 101. Fluid is pumped into an injection tube 124 from action of the cassette 121 as applied by the actuator 123 of the pump 101. A male luer 126, hydraulically communicating with the catheter supply lumen 114, via the injection tube 124, is configured to attach to the female luer 113 of the y-connector 110.

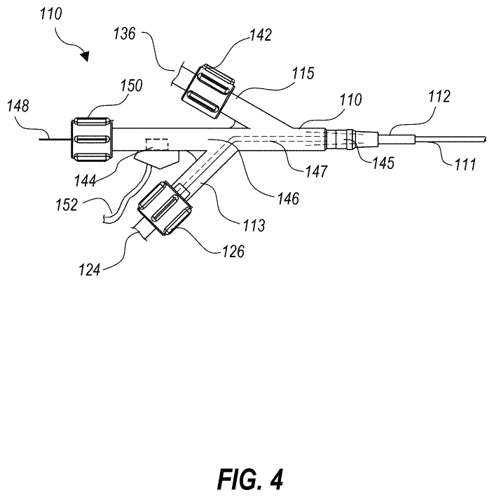

Accessories are illustrated that are intended for applying a vacuum source, such as a syringe 130 having a plunger 132 and a barrel 134, to the aspiration lumen 106 of the catheter 102. The syringe 130 is attached to a vacuum line 136 via the luer 140 of the syringe 130. A stopcock 138 may be used on the luer 140 to maintain the vacuum, or alternatively, the plunger 132 may be a locking variety of plunger that is configured to be locked in the retracted (vacuum) position. A male luer 142 at the end of the vacuum line 136 may be detachably secured to the female luer 115 of the y-connector 110 of the aspiration catheter 102. As shown in more detail in FIG. 4, a pressure sensor or transducer 144 is secured inside an internal cavity 146 of the y-connector 110 proximal to the female luer 113 and the female luer 115. A valve 150, for example a Touhy-Borst, at the proximal end of the y-connector 110 allows hemostasis of the guidewire lumen/aspiration lumen 106 around a guidewire 148. In other embodiments, the valve 150 may comprise a longitudinally spring-loaded seal. The guidewire 148 may be inserted entirely through the guidewire lumen/aspiration lumen 106. Signals output from the pressure sensor 144 are carried through a cable 152 to a connector 154. The connector 154 is plugged into a socket 156 of the pump 101. Pressure related signals may be processed by a circuit board 158 of the pump 101. The pressure transducer 144 may be powered from the pump 101, via the cable 152. The accessories may also be supplied sterile to the user.

A foot pedal 160 is configured to operate a pinch valve 162 for occluding or opening the vacuum line 136. The foot pedal 160 comprises a base 164 and a pedal 166 and is configured to be placed in a non-sterile area, such as on the floor, under the procedure table/bed. The user steps on the pedal 166 causing a signal to be sent along a cable 168 which is connected via a plug 170 to an input jack 172 in the pump 101. The vacuum line 136 extends through a portion of the pump 101. The circuit board 158 of the pump may include a controller 174 configured to receive one or more signals indicating on or off from the foot pedal 160. The controller 174 of the circuit board 158 may be configured to cause an actuator 176 carried by the pump 101 to move longitudinally to compress and occlude the vacuum line 136 between an actuator head 178 attached to the actuator 176 and an anvil 180, also carried by the pump 101. By stepping on the pedal 166, the user is able to thus occlude the vacuum line 136, stopping the application of a negative pressure. In some embodiments, as the pedal 166 of the foot pedal 160 is depressed, the controller may be configured to open the pinch valve 162.

The pressure transducer 144 thus senses a negative pressure and sends a signal, causing the controller to start the motor of the pump 101. As the effect via the electronics is substantially immediate, the motor starts pumping almost immediately after the pedal 166 is depressed. As the pedal 166 of the foot pedal 160 is released, the controller 174 then causes the pinch valve 162 to close. The pressure transducer 144 thus senses that no negative pressure is present and the controller 174 causes the motor of the pump 101 to shut off. Again, the effect via the electronics is substantially immediate, and thus the motor stops pumping almost immediately after the pedal 166 is depressed. During sterile procedures, the main interventionalist is usually “scrubbed” such that the hands only touch items in the sterile field. However, the feet/shoes/shoe covers are not in the sterile field. Thus again, a single user may operate a switch (via the pedal 166) while also manipulating the catheter 102 and guidewire 148. However, this time, it is the sterile field hands and non-sterile field feet that are used. Alternatively, the foot pedal 160 may comprise two pedals, one for occlude and one for open. In an alternative foot pedal embodiment, the pedal 166 may operate a pneumatic line to cause a pressure activated valve or a cuff to occlude and open the vacuum line 136, for example, by forcing the actuator head 178 to move. In another alternative embodiment, the pedal 166 may turn, slide, or otherwise move a mechanical element, such as a flexible pull cable or push rod that is coupled to the actuator 176, to move the actuator head 178. The cable 168 may be supplied sterile and connected to the base 164 prior to a procedure. The occlusion and opening of the vacuum line 136 thus acts as an on and off switch for the pump 101 (via the pressure sensor 144). The on/off function may thus be performed by a user whose hands can focus on manipulating sterile catheters, guidewires, and accessories, and whose foot can turn the pump on and off in a non-sterile environment. This allows a single user to control the entire operation or the majority of operation of the system 100 for aspirating thrombus. This can be an advantage in terms of a rapid, synchronized procedure, but is also helpful in laboratories where additional assistants are not available. The actuator 176 and anvil 180 may be controlled to compress the vacuum line 136 with a particular force, and the actuator 176 may be controlled to move at a particular speed, either when compressing or when removing compression. Speed and force control allows appropriate response time but may also be able to add durability to the vacuum line 136, for example, by not over-compressing. The foot pedal 160 may communicate with the pinch valve 162 via a wired connection through the pump 101 or may communicate with the pinch valve 162 wirelessly. Additionally, or alternatively, the pump may be controlled by buttons 184 or other user interfaces.

It should be noted that in certain embodiments, the pinch valve 162 and the foot pedal 160 may be incorporated for on/off operation of the pinch valve 162 on the vacuum line 136, without utilizing the pressure sensor 144. In fact, in some embodiments, the pressure sensor 144 may even be absent from the system 100 for aspirating thrombus, the foot pedal 160 being used as a predominant control means.

Turning to FIG. 3, a supply tube 186, which contains the catheter supply lumen 114, freely and coaxially extends within the over-the-wire guidewire lumen/aspiration lumen 106. At least a distal end 188 of the supply tube 186 is secured to an interior wall 190 of the guidewire lumen/aspiration lumen 106 of the catheter shaft 111 by adhesive, epoxy, hot melt, thermal bonding, or other securement modalities. A plug 192 is secured within the catheter supply lumen 114 at the distal end 188 of the supply tube 186. The plug 192 blocks the exit of pressurized fluid, and thus the pressurized fluid is forced to exit through an orifice 194 in the wall 196 of the supply tube 186. The free, coaxial relationship between the supply tube 186 and the catheter shaft 111 along their respective lengths, allows for improved flexibility. In some embodiments, in which a stiffer proximal end of the aspiration catheter 102 is desired (e.g., for pushability or even torquability), the supply tube 186 may be secured to the interior wall 190 of the guidewire lumen/aspiration lumen 106 of the catheter shaft 111 along a proximal portion of the aspiration catheter 102, but not along a distal portion. This may be appropriate if, for example, the proximal portion of the aspiration catheter 102 is not required to track through tortuous vasculature, but the distal portion is required to track through tortuous vasculature. The free, substantially unconnected, coaxial relationship between the supply tube 186 and the catheter shaft 111 along their respective lengths, may also be utilized to optimize flow through the guidewire lumen/aspiration lumen 106, as the supply tube 186 is capable of moving out of the way due to the forces of flow (e.g., of thrombus/saline) over its external surface, such that the remaining inner luminal space of the guidewire lumen/aspiration lumen 106 self-optimizes, moving toward the lowest energy condition (least fluid resistance) or toward the largest cross-sectional space condition (e.g., for accommodating and passing pieces of thrombus).

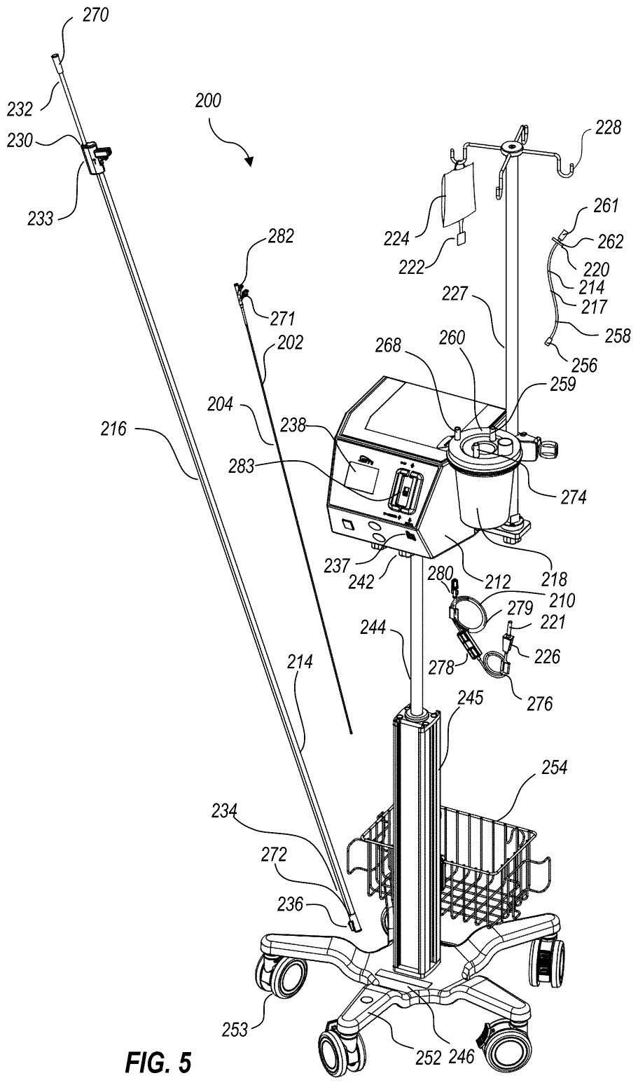

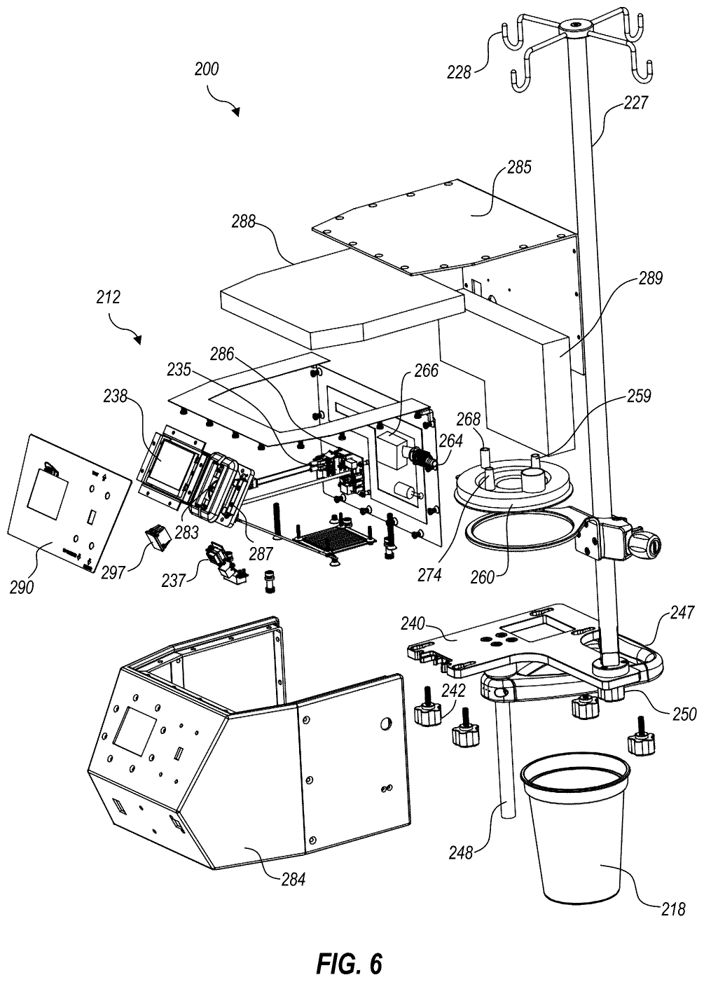

A system for aspirating thrombus 200 is illustrated in FIGS. 5-6. An aspiration catheter 202 is similar to the aspiration catheter 102 of FIGS. 1-4. The aspiration catheter 202 is configured for aspirating thrombus from peripheral vessels, but may also be configured with a size for treating coronary, cerebral, pulmonary or other arteries, or veins. The aspiration catheter 202/system 200 may be used in interventional procedures, but may also be used in surgical procedures. The aspiration catheter 202/system 200 may be used in vascular procedures, or non-vascular procedures (other body lumens, ducts, or cavities). The catheter 202 comprises an elongate shaft 204 configured for placement within a blood vessel of a subject. The catheter 202 may also comprise a catheter supply lumen 114 (FIG. 3) and a guidewire/aspiration lumen 106, each extending along the shaft. The supply lumen 114 may have a proximal end 147 and a distal end 185, and the aspiration lumen 106 may have a proximal end 145 (FIG. 4) and an open distal end 107 (FIG. 3). An orifice or opening 194 may exist at or near the distal end 185 of the supply lumen 114. The orifice or opening 194 may be configured to allow the injection of pressurized fluid into the aspiration lumen 106 at or near the distal end 107 of the aspiration lumen 106 when the pressurized fluid is pumped through the supply lumen 114. In some embodiments, the orifice or opening 194 may be located proximal to the distal end 185 of the supply lumen 114. In some embodiments, the distal end 185 of the supply lumen 114 may comprise a plug 192.

Although examples provided herein focus, in at least some respects, on an aspiration catheter 102 that includes a single orifice 194, an aspiration catheter of a system (e.g., 100 or 200) can include any quantity of orifices through which fluid may pass to form any quantity of fluid jets. For example, a system 100 or 200 for aspirating thrombus can include a quantity of orifices (for forming fluid jets) within a range of 1 to 10, or within a range of 2 to 8, or within a range of 3 to 6.

Although FIG. 3 illustrates the orifice 194 formed in the wall 196 of the supply tube 186, an orifice for forming a fluid jet for an aspiration catheter of a system (e.g., 100 or 200) can be positioned on one or more additional components that are in fluid communication with the supply tube 186. By way of illustrative example, one or more orifices may be formed on a ring (or annulus) that is connected to the catheter shaft 111 (e.g., coaxially connected to the catheter shaft 111 at or near the open distal end 107). The ring can include an internal cavity in fluid communication with the supply tube 186, allowing fluid to travel through the supply tube 186 and the internal cavity of the ring for jetting through the orifice(s) of the ring.

Furthermore, FIG. 3 illustrates the orifice 194 formed as a radial opening in the wall 196 of the supply tube 186, with the orifice wall(s) that forms the orifice 194 between inner and outer surfaces of the supply tube 186 being perpendicular to the tangential axis of the wall 196. In some embodiments, an orifice for forming a fluid jet for an aspiration catheter of a system (e.g., 100 or 200) may be formed as an angled opening, where the orifice wall(s) that forms the orifice (between inner and outer surfaces of a material) has an acute or obtuse angle relative to the tangential axis of the material on which the orifice is formed (e.g., material of a supply tube or an additional component connected to the supply tube). For example, the angle between the orifice wall(s) and the tangential axis of the material on which the orifice is formed can be within a range of 20° to 70°, or 30° to 60°, or 40° to 50° (or within their complementary ranges).

The orifice(s) of an aspiration catheter 102 of a system (e.g., 100 or 200) for providing fluid jets (i.e., “jet-forming” orifices) may be implemented with various sizes or shapes in different embodiments. In one example, an orifice may comprise a circular hole with a diameter between 0.03 mm (0.001 inches) and 0.15 mm (0.006 inches), or between about 0.0508 mm (0.002 inches) and about 0.1016 mm (0.004 inches), or about 0.0787 mm (0.0031 inches). The diameter of the supply lumen 114 may be between about 0.3048 mm (0.012 inches) and about 0.4826 mm (0.019 inches), or between about 0.3556 mm (0.014 inches and about 0.4318 mm (0.017 inches), or about 0.3937 mm (0.0155 inches). As another example, an orifice may comprise a rectangular hole with each side thereof having a length between 0.02 mm (0.0008 inches) and 0.20 mm (0.008 inches). In some implementations, the total cross-sectional area of all jet-forming orifices of an aspiration catheter of a system is between 0.002 mm\hat{ }2 and 0.02 mm\hat{ }2, or between 0.003 mm\hat{ }2 and 0.015 mm\hat{ }2, or between 0.005 mm\hat{ }2 and 0.01 mm\hat{ }2. In some embodiments, pressure measured at the deliver location of each jet-forming orifice during operation of the system (e.g., 100 or 200) is between 400 psi (2.6 MPa) and 2,000 psi (13.8 MPa), or between 500 psi (3.4 MPa) and 2,000 psi (13.8 MPa), or between 600 psi (4.1 MPa) and 1,750 psi (12.1 MPa).

In some implementations, the diameter of the aspiration lumen 106 is within a range of 2 mm (0.08 inches) to 4 mm (0.16 inches). The orifice 194 may be set proximally of the open distal end 107 by a set amount. For example, orifice 194 can be set proximally of the open distal end 107 by about 0.040″ (inches), and in one configuration by 0.051″+−0.003″ or by another desired amount. For example, orifice 42 can be set proximally of the open distal end 107 by approximately 0.035″, 0.036″, 0.037″, 0.038″, 0.039″, 0.040″, 0.041″, 0.042″, 0.043″, 0.044″, 0.045″, 0.046″, 0.047″, 0.048″, 0.049″, 0.050″, 0.051″, 0.052″, 0.053″, 0.054″, 0.055″, 0.056″, 0.057″, 0.058″, 0.059″, 0.060″, or a range defined by any two of the foregoing. In still other configurations, the open distal end 107 can be set proximally of the open distal end 107 by about 0.01″ to about 50″.

In still another configuration a diameter of the aspiration lumen 106 is about 0.075″ (inches) to about 0.177″ (inches), the orifice 194 (and so a distal end of the supply tube 186) can be set proximally of open distal end 107 about 12″ (inches). In still another configuration a diameter of the aspiration lumen 106 is about 0.075″ (inches), the orifice 194 (and so a distal end of the supply tube 186) can be set proximally of open distal end 107 about 0.035″ (inches) to about 0.060″ (inches). In still another configuration, the orifice 194 (and so a distal end of the supply tube 186) can be set proximally of open distal end 107 about 0.010″ (inches) to about 50″ (inches) where the catheter 102 has a catheter size ranging from about 3 Fr to about 50 Fr. In still another configuration, the orifice 194 (and so a distal end of the supply tube 186) can be set proximally of open distal end 107 about 0.010″ (inches) to about 0.200″ (inches), from about 0.010″ (inches) to about 2″ (inches), 0.010″ (inches) to about 40″ (inches), 0.010″ (inches) to about 50″ (inches), or a range defined by any two of the foregoing. In still other configurations, the orifice 194 (and so a distal end of the supply tube 186) can be set proximally of open distal end 107 based upon Table 1:

| | Distal Orifice Proximal Offset |
|---|---|
| Catheter size | (range in inches) |
| 3 Fr | .01″ to .600″ |
| 4 Fr | |
| 5 Fr | |
| 6 Fr | |
| 7 Fr | |
| 8 Fr | |
| 9 Fr | 0.01″ to 40″ |
| 10 Fr | |
| 11 Fr | |
| 12 Fr | .01″ to 40″ |
| 13 Fr | |
| 14 Fr | |
| 15 Fr | |
| 16 Fr | |
| 17 Fr | .01″ to 50″ |
| 18 Fr | |
| 19 Fr | |
| 20 Fr | |
| 21 Fr | |
| 22 Fr | |
| 23 Fr | |
| 24 Fr | |
| 25 Fr | |
| 26 Fr | |

In another configuration, the orifice 194 (and so a distal end of the supply tube 186) can be set proximally of open distal end 107 about 0.010″ (inches) to about 0.200″ (inches) for a catheter having a catheter size ranging from about 3 Fr to about 8 F. In another configuration, the orifice 194 (and so a distal end of the supply tube 186) can be set proximally of open distal end 107 about 0.010″ (inches) to about 2″ (inches) for a catheter having a catheter size ranging from about 9 Fr to about 11 Fr. In another configuration, the orifice 194 (and so a distal end of the supply tube 186) can be set proximally of open distal end 107 about 0.010″ (inches) to about 40″ (inches) for a catheter having a catheter size ranging from about 12 Fr to about 16 F. In another configuration, the orifice 194 (and so a distal end of the supply tube 186) can be set proximally of open distal end 107 about 0.010″ (inches) to about 50″ (inches) for a catheter having a catheter size ranging from about 17 Fr to about 26 F. In other configurations, the ranges can include any combination of a range defined by any two of the foregoing proximal location of the orifice 194 with a range defined by any two of the foregoing catheter sizes.

A pump set 210 (e.g., tubing set) is configured to hydraulically couple the supply lumen 114 to a pump within a saline drive unit (SDU) 212, for injecting pressurized fluid (e.g., saline, heparinized saline) through the supply lumen 114. Suction tubing 214, comprising sterile suction tubing 216 and non-sterile suction tubing 217, is configured to hydraulically couple a vacuum canister 218 to the aspiration lumen 106. A filter 220 may be carried in-line on the suction tubing 214, for example, connected between the sterile suction tubing 216 and the non-sterile suction tubing 217, or on the non-sterile suction tubing 217. The filter 220 is configured to capture large elements such as large pieces of thrombus or emboli.

The pump set 210 includes a saline spike 221 for connection to a port 222 of a saline bag 224, and an inline drip chamber 226 for visually assessing the movement of saline, as well as keeping air out of the fluid being injected. The saline bag 224 may be hung on an IV pole 227 on one or more hooks 228. A pressure sensor 230 such as a vacuum sensor may be used within any lumen of the pump set 210, the suction tubing 214, the supply lumen 114 or aspiration lumen 106 of the catheter 202, or any other component which may see fluid flow. Additional or alternative pressure sensors may be implemented to measure pressure associated with the vacuum canister 218. The pressure sensor 230 is shown in FIG. 5 within a lumen at a junction between a first aspiration tube 232 and a control 233. A cable 234 carries signals output from the pressure sensor 230 to a controller 235 in the SDU 212. A connector 236, electrically connected to the cable 234, is configured to be detachably coupled to a mating receptacle 237 (e.g., input jack) in the SDU 212. The SDU 212 also may have a display 238, including an LCD screen or alternative screen or monitor, in order to visually monitor parameters and status of a procedure. In some instances, one or more fluid flow sensors is/are utilized in addition to or as an alternative to the pressure sensor 230. In some embodiments, the fluid flow sensor is a Doppler flow velocity sensor, or other type of flow sensor. In some instances, flow metrics may be inferred or characterized by implementing multiple pressure sensors (e.g., (i) a pressure sensor on the pump set 210, suction tubing 214, or aspiration lumen 106, and (ii) a pressure sensor on the vacuum canister 218).

In the example of FIGS. 5 and 6, the SDU 212 is held on a mount 240 by four locking knobs 242. The mount 240 is secured to a telescoping rod 244 that is adjustable from a cart base 245 via a cart height adjustment knob or other element 246. The mount 240 and a handle 247 are secured to the rod 244 via an inner post 248 that is insertable and securable within an inner cavity in the rod 244. The IV pole 227 secures to the mount 240 via a connector 250. The base 245 may include legs 252 having wheels 253 (e.g., three or more wheels or four or more wheels) and may be movable via the handle 247. The system 200 may also carry a basket 254 for placement of components, products, documentation, or other items.

In use, a user connects a first connector 256 at a first end 258 of the non-sterile suction tubing 217 to a port 259 on the lid 260 of the canister 218, and connects a second connector 261 at a second end 262 of the non-sterile suction tubing 217 to a vacuum pump input 264 in the SDU 212. A vacuum pump 266 may be carried within the SDU 212 in order to maintain a vacuum/negative pressure within the canister 218. Alternatively, the vacuum inside the canister 218 may be maintained manually, without a vacuum pump, by evacuating the canister 218 via one or more additional ports 268. A user connects a first connector 270 of the sterile suction tubing 216 to an aspiration luer 271 of the aspiration catheter 202 (similar to luer 115), and connects the second connector 272 of the sterile suction tubing 216 to port 274 in the lid 260 of the canister 218. Connector 236 is then coupled to the mating receptacle 237 in the SDU 212 for communication with the control 233 and/or the pressure sensor 230. For instance, the connector 236 can be snapped into mating receptacle 237 in the SDU 212 for communication with elements of the control 233 and/or for communication with the pressure sensor 230, either via cable 234, and/or additional cables or wires. Alternatively, the connector 236 may couple to the mating receptacle 237 by clipping, friction fitting, vacuum fitting, or other means.

After allowing saline to purge through the supply tube 276, cassette 278, and injection tube 279 of the pump set 210, the user connects the luer connector 280 of the pump set 210 to a luer 282 of the aspiration catheter 202 (similar to luer 113). The cassette 278 (similar to cassette 121) is then attached to a saddle 283 in the SDU 212. The saddle 283 is configured to reciprocate a piston to inject the saline from the IV bag 224 at high pressure, after the cassette 278 is snapped in place, keeping the internal contents (e.g., saline) sterile. Systems configured for performing this type of sterile injection of high-pressure saline are described in U.S. Pat. No. 9,883,877, issued Feb. 6, 2018, and entitled, “Systems and Methods for Removal of Blood and Thrombotic Material”, which is incorporated by reference in its entirety for all purposes. The SDU 212 is enclosed within a case 284 and a case lid 285. The controller 235 may reside on a circuit board 286. Noise from a motor 287 controlling the saddle 283 and from the vacuum pump 266 may be abated by internal foam sections 288, 289. The saddle 283 may be moved directly by the motor 287, or may be moved with pneumatics, using a cycled pressurization. An interface panel 290 provides one or more switches 297 and the display 238. Alternatively, the cassette 121 may couple to the saddle 283 by clipping, friction fitting, vacuum fitting, or other means.

### Clot Engagement Detection in Thrombectomy Systems

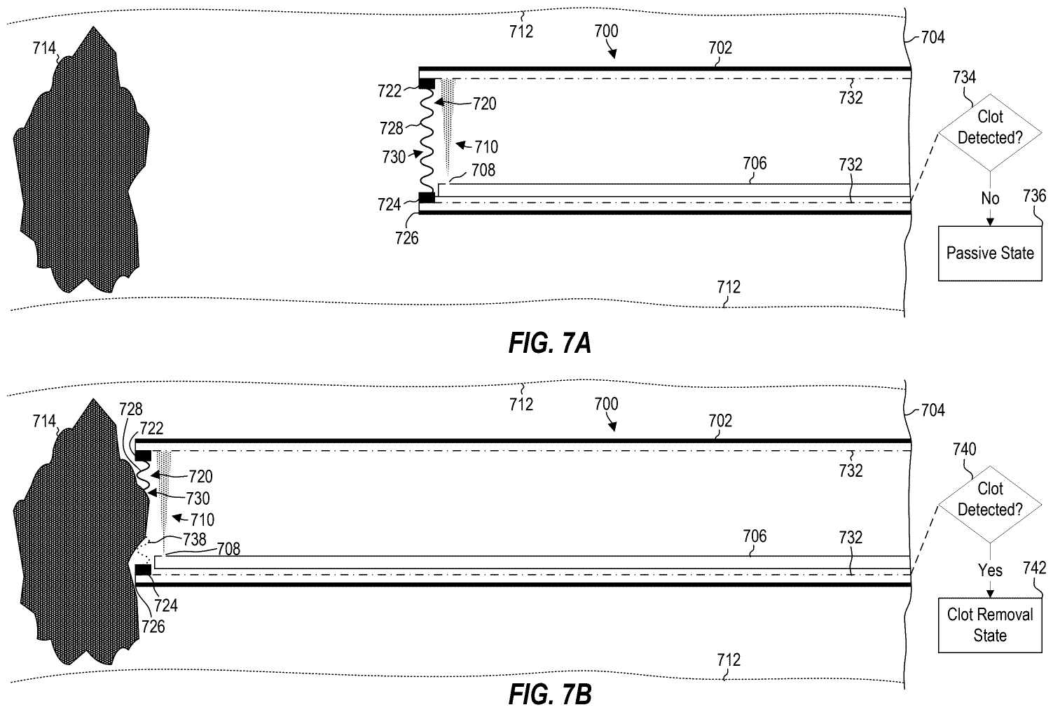

FIGS. 7A and 7B illustrate conceptual representations of components of a thrombectomy system 700. FIGS. 7A and 7B depict a distal portion of an aspiration catheter 702 of the thrombectomy system 700 (break lines 704 separate the distal portion of the aspiration catheter 702 from other portions thereof and from other components of the thrombectomy system 700). The aspiration catheter 702 of FIGS. 7A and 7B includes a supply lumen 706 with an orifice 708 configured to release a high-pressure saline jet 710 at or near a distal opening of the aspiration catheter 702 (e.g., as controlled by an SDU of the thrombectomy system).

The aspiration catheter 702 of FIGS. 7A and 7B is configured for advancement through vasculature of a subject (e.g., a human patient) to facilitate clot removal therefrom. FIGS. 7A and 7B conceptually depict vessel walls 712 through which the aspiration catheter 702 may advance and from which a clot 714 may be removed. For instance, upon advancing into engagement with the clot 714, a clot removal state of the thrombectomy system 700 may be activated. The clot removal state may cause the saline jet 710 to macerate the clot 714 and may cause negative pressure to be applied at the distal opening of the aspiration catheter 702 (e.g., by operation of one or more vacuum pumps/motors of the thrombectomy system 700) to aspirate the clot 714 and/or fragments thereof from the subject vasculature.

As noted above, practitioners/users may experience difficulty in determining whether the distal opening of the aspiration catheter 702 is sufficiently near the clot 714 to cause removal of the clot 714 when the clot removal state is activated. Accordingly, the thrombectomy system 700 of FIGS. 7A and 7B includes a clot detection system 720. In the example of FIGS. 7A and 7B, the clot detection system 720 includes an emitter 722 and a detector 724, which are positioned proximate to the distal end 726 of the aspiration catheter 702. As used herein, “proximate” refers to a close distance between two elements, such as a distance less than about 10 cm, less than about 9 cm, less than about 8 cm, less than about 7 cm, less than about 6 cm, less than about 5 cm, less than about 4 cm, less than about 3 cm, less than about 2 cm, less than about 1 cm, less than about 7.5 mm, less than about 5 mm, less than about 2.5 mm, less than about 1 mm, or a distance within a range that has end points within any of the foregoing ranges. FIGS. 7A and 7B depict the emitter 722 and the detector 724 positioned on an inner wall of the distal opening of the aspiration catheter 702, with the emitter 722 and the detector 724 being offset from one another about the inner wall of the distal opening by about 180°. Other positional configurations of the emitter 722 and the detector 724 on the aspiration catheter 702 are within the scope of the present disclosure (e.g., being offset by 90°-180°, being positioned on the distal-facing wall of the distal opening, etc.)

In the example of FIG. 7A, the emitter 722 is configured to emit a signal 728 along a propagation path 730 toward the detector 724. The detector 724 is configured to detect the signal 728 of the emitter 722. The emitter 722 and the detector 724 may be connected to fiber optic 732 or other communication cabling/means that extend proximally from the emitter 722 and the detector 724 within the aspiration catheter 702. The fiber optics 732 may facilitate signal transmission/communication between the clot detection system 720 and other control systems of the thrombectomy system 700. For instance, the signal 728 may be generated by a component of the thrombectomy system 700 (not shown in FIG. 7A) and propagated toward the emitter 722 via the fiber optics 732 (or other communication cabling/means). The signal detected by the detector 724 (if any) may be propagated toward control/processing components of the thrombectomy system 700 (not shown in FIG. 7A) via the fiber optics 732 (or other communication cabling/means).

In some implementations, the signal detected by the detector 724, or lack thereof, may be used to determine whether the propagation path 730 is occluded, which may indicate the presence or absence of clot material in the propagation path 730 (or otherwise proximate to the distal end 726 of the aspiration catheter 702). For instance, FIG. 7A depicts an instance where the aspiration catheter 702 has not reached the clot 714. Accordingly, in the example of FIG. 7A, the signal 728 emitted by the emitter 722 may be substantially uninterrupted by the clot 714, enabling the detector 724 to detect the uninterrupted signal 728. The substantially uninterrupted signal 728 may comprise a pattern signal (or even an intermittent signal) associated with propagation through blood (and/or another propagation medium, such as saline released from the thrombectomy system 700) in the absence of clot material. Detection of the uninterrupted signal 728 by the detector 724 may indicate that the aspiration catheter 702 is not engaged with the clot 714. The signal detected by the detector 724 may be processed by processing/control components of the thrombectomy system 700 (not shown in FIG. 7A) to determine whether clot absence is indicated by the detected signal.

In some implementations, the thrombectomy system 700 may selectively remain in a passive state in response to determining that the signal detected by the detector 724 indicates that the aspiration catheter 702 is not engaged with a clot (as indicated in FIG. 7A by decision block 734 and the “No” arrow extending therefrom toward action block 736). The passive state may comprise a state in which aspiration components of the thrombectomy system 700 (e.g., the vacuum pump/motor, SDU, etc.) refrain from generating negative pressure at the distal portion of the aspiration catheter or only generate a low level of negative pressure thereat (e.g., pursuant to a low-level aspiration state).

FIG. 7B depicts an instance where the aspiration catheter 702 has reached the clot 714. Accordingly, in the example of FIG. 7B, the signal 728 emitted by the emitter 722 is at least partially interrupted, interfered with, and/or blocked by the clot 714, causing the detector 724 to fail to detect the signal 728 emitted by the emitter 722 or to detect a weakened/altered signal 738. For instance, the clot 714 may cause the magnitude or temporal characteristics of the signal 728 to change, resulting in the weakened/altered signal 738. Failure to detect the signal 728 (or detection of the weakened/altered signal 738) by the detector 724 may indicate that the aspiration catheter 702 is engaged with the clot 714. The weakened/altered signal 738 may comprise a pattern signal associated with a clot or clot type. For instance, acute, subacute, or chronic clots may alter the signal 728 in different ways, causing the weakened/altered signal 738 to take on different pattern forms. The signal (or lack of a signal) detected by the detector 724 may be processed by processing/control components of the thrombectomy system 700 (not shown in FIG. 7B) to determine whether clot presence/engagement is indicated by the detected signal. In some instances, processing of the signal detected by the detector 724 may allow the system to determine the clot type, if present (based on the pattern(s) in the detected signal), which the system may present as output to a user.

In some implementations, the thrombectomy system 700 may selectively activate (or remain in) a clot removal state in response to determining that the signal (or lack of signal) detected by the detector 724 indicates that the aspiration catheter 702 is engaged with a clot (as indicated in FIG. 7B by decision block 740 and the “Yes” arrow extending therefrom toward action block 742). The clot removal state may comprise a state in which aspiration components of the thrombectomy system 700 (e.g., the vacuum pump/motor, SDU, etc.) generate negative pressure at the distal portion of the aspiration catheter 702 to facilitate clot aspiration.

In some embodiments, after activation of the clot removal state, the signal (or lack of signal) detected by the detector 724 may be continuously monitored by components of the thrombectomy system 700. In this way, during operation of the clot removal state, the thrombectomy system 700 may determine when the detector 724 again begins to detect a substantially uninterrupted signal 728 emitted by the emitter 722, which may indicate that clot maceration/aspiration has been completed (e.g., indicating that a clot is no longer proximate to the distal end 726 of the aspiration catheter 702). In response to determining that the detector again detects the uninterrupted signal 728 (during operation of the clot removal state), the thrombectomy system 700 may selectively return to the passive state.

The functionality described hereinabove with reference to FIGS. 7A and 7B may enable thrombectomy systems to (i) selectively activate (or remain in) clot removal states in response to detecting clot material proximate to the distal end of the aspiration catheter and (ii) selectively deactivate (or refrain from) clot removal states in response to detecting that clot material is not proximate to the distal end of the aspiration catheter. Such techniques may advantageously reduce operation times, reduce patient blood loss, reduce damage to vessel walls, etc.

The signal 728 emitted by the emitter 722 may take on various forms. For example, signal/emissions of the emitter 722 may comprise light emissions/signals, such as visible spectrum light, near-infrared (NIR) light, and/or other light spectra (e.g., UV light, IR light). In some implementations, the emitter 722 is configured to emit sound emissions. The wave characteristics of the signal detected by the detector 724 (e.g., compared to wave characteristics of the signal 728 emitted by the emitter 722) may indicate whether the distal end 726 of the aspiration catheter 702 is un-occluded or proximate to blood, clot material, a vessel wall, etc.

In some implementations, the thrombectomy system 700 is configured to disburse a propagation medium toward the propagation path 730 to enable or improve propagation of the signal 728 through the propagation path 730 from the emitter 722 to the detector 724. For instance, the orifice 708 of the supply lumen 706 may be configured such that at least some fluid released or jetted therefrom is directed toward the propagation path 730, thereby enabling the fluid to operate as a propagation medium for the signal 728 (when the propagation path 730 is un-occluded). Additional or alternative fluid delivery mechanisms may be employed to disperse a propagation medium toward the propagation path 730, such as a dedicated supply line.

In some implementations, the signal 728 emitted by the emitter comprises an electrical signal. In such instances, the emitter 722 and the detector 724 of the clot detection system 720 may be regarded as an anode and cathode pair. In such an example, the clot detection system 720 may apply the signal 728 in the form of a fixed voltage current via the anode and the cathode. The current and/or resistance detected during application of the fixed voltage current may be indicative of the presence or absence of clot material proximate to the distal end 726 of the aspiration catheter 702 (e.g., by comparison to reference values associated with clot presence and/or absence). When the measured current and/or resistance indicate clot presence, the thrombectomy system 700 may activate or remain in the clot removal state, whereas when the measured current and/or resistance indicate clot absence, the thrombectomy system 700 may enter or remain in the passive state. In one example, the anode and the cathode can be connected via a resistor along the boundary of the wall of the aspiration catheter 702. The resistor can comprise a higher resistance than clot material expected to be encountered by the aspiration catheter 702. When a clot lodges in the mouth of the aspiration catheter 702, the clot can provide the anode/cathode pair with a lower resistance pathway, resulting in a measurable impedance drop that can indicate clot engagement.

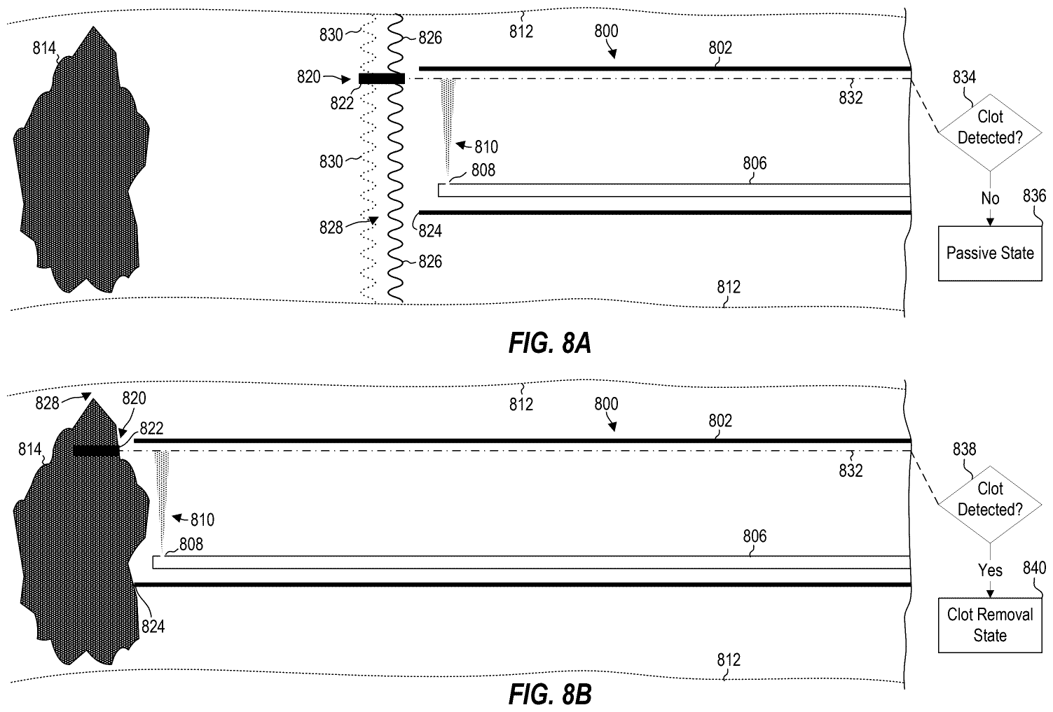

FIGS. 8A and 8B illustrate conceptual representations of a thrombectomy system 800 with a clot detection system 820 that implements a clot detection probe 822 (or simply “detection probe”). The thrombectomy system 800 includes components similar to thrombectomy system 700, such as an aspiration catheter 802, a supply lumen 806 with an orifice 808 for releasing a saline jet 810, etc.

In the example of FIGS. 8A and 8B, the detection probe extends distally from the distal end 824 of the aspiration catheter 802 (e.g., from a distal opening of the aspiration catheter 802). The detection probe 822 of the clot detection system 820 is configured to emit a signal 826 along a propagation path 828 extending between the detection probe 822 and the vessel lumen/walls. The signal 826 may reflect off of the vessel walls 812 (resulting in a reflected signal 830). In some implementations, the detection probe 822 is configured to detect the reflected signal 830. The detection probe 822 may be connected to fiber optic 832 or other communication cabling/means that extend proximally from the detection probe 822 into the aspiration catheter 802. The fiber optics 832 may facilitate signal transmission/communication between the detection probe 822 and other control systems of the thrombectomy system 800. For instance, the signal 826 may be generated by a component of the thrombectomy system 800 (not shown in FIG. 8A) and propagated toward the detection probe 822 via the fiber optics 832 (or other communication cabling/means). The signal detected by the detection probe 822 (if any) may be propagated toward control/processing components of the thrombectomy system 800 (not shown in FIG. 8A) via the fiber optics 832 (or other communication cabling/means).

The signal detected by the detection probe 822 (or lack thereof) may be used to determine whether clot material is proximate to the distal end 824 of the aspiration catheter 802 (e.g., to determine whether clot material occludes or occupies the propagation path 828). For example, as shown in FIG. 8A, the signal 826 may freely propagate from the detection probe 822 along the propagation path 828 and reflect off of the vessel walls 812 for detection by the detection probe 822 when a clot 814 is absent from the propagation path 828. Accordingly, in the example of FIG. 8A, the signal 826 emitted by the detection probe 822 may be substantially uninterrupted by the clot 814, enabling the detection probe 822 to detect the reflected signal 830. Detection of the reflected signal 830 by the detection probe 822 may indicate that no clot material is present in the propagation path 828 (e.g., may indicate that the aspiration catheter 802 is not proximate to or engaged with the clot 814). The signal detected by the detection probe 822 may be processed by processing/control components of the thrombectomy system 800 to determine whether clot absence is indicated by the detected signal.

In some implementations, the thrombectomy system 800 may selectively remain in a passive state in response to determining that the signal detected by the detection probe 822 indicates that the aspiration catheter 802 is not engaged with a clot (as indicated in FIG. 8A by decision block 834 and the “No” arrow extending therefrom toward action block 836). The passive state may comprise a state in which aspiration components of the thrombectomy system 800 (e.g., the vacuum pump/motor, SDU, etc.) refrain from generating negative pressure at the distal portion of the aspiration catheter or only generate a low level of negative pressure thereat (e.g., pursuant to a low-level aspiration state).

As shown in FIG. 8B, the signal 826 may be unable to freely propagate from the detection probe 822 along the propagation path 828 and reflect off of the vessel walls 812 for detection by the detection probe 822 when a clot 814 is present in the propagation path 828. For instance, the clot 814 may at least partially interrupt, interfere with, and/or block the signal 826 emitted by the detection probe 822, causing the detection probe 822 to fail to detect a reflected signal or to detect a weakened/altered reflected signal. Failure to detect a reflected signal (or detection of the weakened/altered signal) by the detection probe 822 may indicate that the aspiration catheter 802 is engaged with the clot 814. The signal (or lack of a signal) detected by the detection probe 822 may be processed by processing/control components of the thrombectomy system 800 to determine whether clot presence/engagement is indicated by the detected signal.

In some implementations, the thrombectomy system 800 may selectively activate (or remain in) a clot removal state in response to determining that the signal (or lack of signal) detected by the detection probe 822 indicates that the aspiration catheter 802 is engaged with a clot (as indicated in FIG. 8B by decision block 838 and the “Yes” arrow extending therefrom toward action block 840). The clot removal state may comprise a state in which aspiration components of the thrombectomy system 800 (e.g., the vacuum pump/motor, SDU, etc.) generate negative pressure at the distal portion of the aspiration catheter 802 to facilitate clot aspiration.

In some embodiments, after activation of the clot removal state, the signal (or lack of signal) detected by the detection probe 822 may be continuously monitored by components of the thrombectomy system 800. In this way, during operation of the clot removal state, the thrombectomy system 800 may determine when the detection probe 822 again begins to detect a reflected signal 830 emitted by the detection probe 822, which may indicate that clot maceration/aspiration has been completed (e.g., indicating that a clot is no longer proximate to the distal end 824 of the aspiration catheter 802). In response to determining that the detection probe 822 again detects the reflected signal 830 (during operation of the clot removal state), the thrombectomy system 800 may selectively return to the passive state.

The functionality described hereinabove with reference to FIGS. 8A and 8B may enable thrombectomy systems to (i) selectively activate (or remain in) clot removal states in response to detecting clot material proximate to the distal end of the aspiration catheter and (ii) selectively deactivate (or refrain from) clot removal states in response to detecting that clot material is not proximate to the distal end of the aspiration catheter. Such techniques may advantageously reduce operation times, reduce patient blood loss, reduce damage to vessel walls, etc.

The detection probe 822 may be configured to emit the signal 826 according to any suitable orientation, coverage, magnitude, etc. The signal 826 may take on various forms. For example, signal/emissions of the detection probe may comprise light emissions/signals, such as visible spectrum light, near-infrared (NIR) light, and/or other light spectra (e.g., UV light, IR light). The detection probe 822 may emit signals according to various imaging or detection modalities, such as, by way of non-limiting example, optical coherence tomography (OCT), light detection and ranging (LiDAR), time-of-flight, and/or others.

In some implementations, the detection probe 822 is configured to emit sound emissions. The wave characteristics of the signal detected by the detection probe 822 (e.g., compared to wave characteristics of the signal 826 emitted by the detection probe 822) may indicate whether the distal end 824 of the aspiration catheter 802 is un-occluded or proximate to blood, clot material, a vessel wall, etc.

In some implementations, the thrombectomy system 800 is configured to disburse a propagation medium toward the propagation path 828 to enable or improve propagation of the signal 826 through the propagation path 828 between the detection probe 822 and the vessel lumen/walls 812. For instance, the orifice 808 of the supply lumen 806 may be configured such that at least some fluid released or jetted therefrom is directed toward the propagation path 828, thereby enabling the fluid to operate as a propagation medium for the signal 826 (when the propagation path 828 is un-occluded). Additional or alternative fluid delivery mechanisms may be employed to disperse a propagation medium toward the propagation path 828, such as a dedicated supply line.

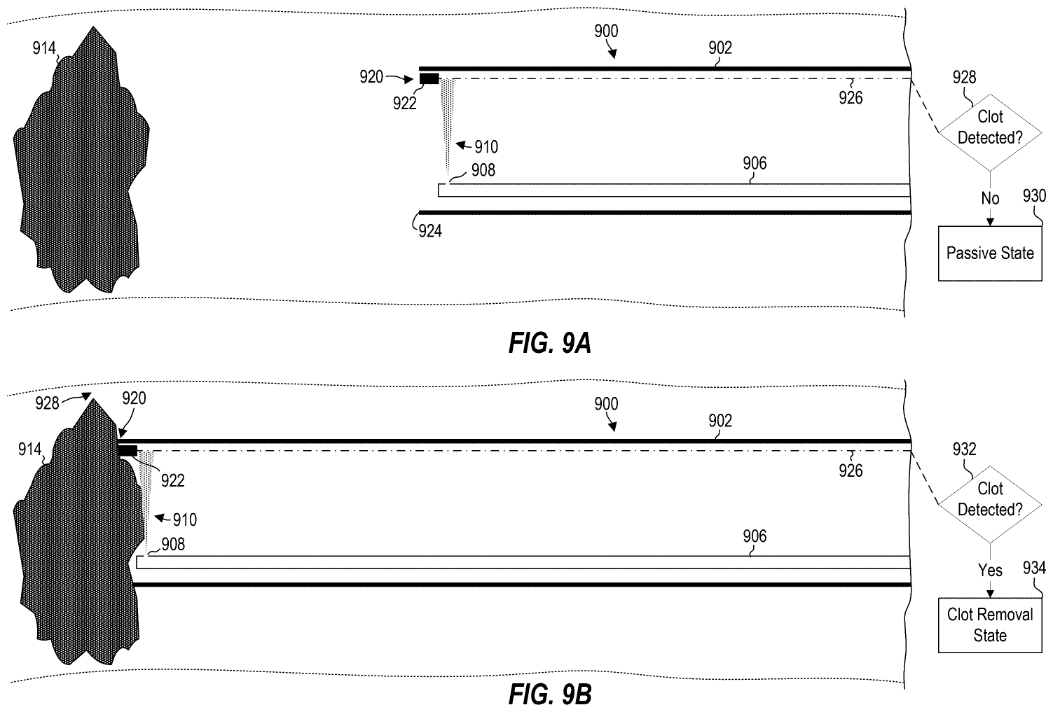

The thrombectomy system 900 of FIGS. 9A and 9B also includes a clot detection system 920 that implements a clot detection probe 922. The thrombectomy system 900 includes components similar to thrombectomy system 800, such as an aspiration catheter 902, a supply lumen 906 with an orifice 908 for releasing a saline jet 910, etc.

In the example of FIGS. 9A and 9B, the detection probe 922 is positioned proximate to the distal end 924 of the aspiration catheter 902 (e.g., on an inner wall of a distal opening of the aspiration catheter 902). The detection probe 922 of the thrombectomy system 900 is configured to detect force or pressure exerted on the detection probe 922. The detection probe 922 may be implemented as a fiber optic force or pressure sensor, or other type of force or pressure sensor (e.g., strain gauge, load cell, piezoelectric, capacitive, magnetic, piezoresistive, resonant, optical, potentiometric, microelectromechanical systems (MEMS), etc.). The detection probe 922 of FIGS. 9A and 9B is connected to fiber optic 926 or other communication cabling/means that extend proximally from the detection probe 922 into the aspiration catheter. The fiber optics 926 may facilitate signal transmission/communication between the detection probe 922 and other control systems of the thrombectomy system 900. The signal output by the detection probe 922 (e.g., brought about by application of force and/or pressure to the detection probe 922) may be propagated toward control/processing components of the thrombectomy system 900 (not shown in FIG. 9A) via the fiber optics 926 (or other communication cabling/means).

The force or pressure detected by the detected probe 922 (or lack thereof) may be used to determine whether clot material is proximate to the distal end 924 of the aspiration catheter 902. For instance, the force or pressure detected by the detection probe 922 failing to satisfy one or more conditions (e.g., one or more threshold force or pressure values or ranges) may indicate that a clot 914 is not proximate to the distal end 924 of the aspiration catheter 902. The thrombectomy system 900 may selectively remain in a passive state in response to determining that the signal output by the detection probe 922 indicates that no clot 914 is proximate to the distal end 924 of the aspiration catheter 902 (as indicated in FIG. 9A by decision block 928 and the “No” arrow extending therefrom toward action block 930). The passive state may comprise a state in which aspiration components of the thrombectomy system 900 (e.g., the vacuum pump/motor, SDU, etc.) refrain from generating negative pressure at the distal portion of the aspiration catheter or only generate a low level of negative pressure thereat (e.g., pursuant to a low-level aspiration state).

In contrast, the force or pressure detected by the detection probe 922 satisfying the one or more conditions may indicate that clot material is proximate to the distal end 924 of the aspiration catheter 902. The thrombectomy system 900 may selectively activate (or remain in) a clot removal state in response to determining that the signal output by the detection probe 922 indicates that a clot 914 is proximate to the distal end 924 of the aspiration catheter 902 (as indicated in FIG. 9B by decision block 932 and the “Yes” arrow extending therefrom toward action block 934). The clot removal state may comprise a state in which aspiration components of the thrombectomy system 900 (e.g., the vacuum pump/motor, SDU, etc.) generate negative pressure at the distal portion of the aspiration catheter 902 to facilitate clot aspiration.

In some embodiments, after activation of the clot removal state, the signal output by the detection probe 922 may be continuously monitored by components of the thrombectomy system 900. In this way, during operation of the clot removal state, the thrombectomy system 900 may determine when the detection probe 922 again begins to detect a force or pressure that fails to satisfy the condition(s) noted above, which may indicate that clot maceration/aspiration has been completed (e.g., indicating that a clot is no longer proximate to the distal end 924 of the aspiration catheter 902). In response to determining that the detection probe 922 again detects such a force or pressure (during operation of the clot removal state), the thrombectomy system 900 may selectively return to the passive state.

The functionality described hereinabove with reference to FIGS. 9A and 9B may enable thrombectomy systems to (i) selectively activate (or remain in) clot removal states in response to detecting clot material proximate to the distal end of the aspiration catheter and (ii) selectively deactivate (or refrain from) clot removal states in response to detecting that clot material is not proximate to the distal end of the aspiration catheter. Such techniques may advantageously reduce operation times, reduce patient blood loss, reduce damage to vessel walls, etc.

In some implementations, a thrombectomy system may analyze data output by a clot detection system (e.g., resistance/current data, detected signal data, force/pressure data) to determine clot characteristics (e.g., clot composition, texture, elasticity, fragility, hardness/stiffness, texture, adherence, shape, etc.). In some instances, the clot characteristics may influence the type of aspiration state implemented by the thrombectomy system.

### EXAMPLE EMBODIMENTS

Embodiments disclosed herein can include those in the following numbered clauses:

Clause 1. A thrombectomy system, comprising: an aspiration catheter configured for advancement through vasculature of a subject to facilitate clot removal from the vasculature of the subject; and a clot detection system comprising an emitter and a detector, wherein the emitter and the detector are positioned proximate to a distal end of the aspiration catheter, wherein the detector is configured to detect emissions of the emitter to indicate whether a propagation path between the emitter and the detector is occluded, wherein occlusion of the propagation path between the emitter and the detector indicates a presence of a clot proximate to the distal end of the aspiration catheter.

Clause 2. The thrombectomy system of clause 1, wherein the emitter and the detector are positioned on an inner wall of a distal opening of the aspiration catheter.

Clause 3. The thrombectomy system of clause 2, wherein the clot detection system further comprises one or more fiber optics extending proximally from the emitter and the detector within the aspiration catheter.

Clause 4. The thrombectomy system of clause 2, wherein the emitter and the detector are offset from one another on the inner wall of the distal opening by about 180°.

Clause 5. The thrombectomy system of clause 1, wherein the emissions of the emitter comprise light emissions.

Clause 6. The thrombectomy system of clause 5, wherein the light emissions comprise visible spectrum light or near-infrared light.

Clause 7. The thrombectomy system of clause 1, wherein the emissions of the emitter comprise sound emissions.

Clause 8. The thrombectomy system of clause 1, wherein the aspiration catheter comprises a jet configured to release a propagation medium toward the propagation path to enable the emissions of the emitter to propagate through the propagation medium when the propagation path is un-occluded.

Clause 9. The thrombectomy system of clause 1, wherein the thrombectomy system is configured to selectively activate a clot removal state after the clot detection system indicates that a clot is proximate to the distal end of the aspiration catheter.

Clause 10. The thrombectomy system of clause 9, wherein, during operation of the clot removal state, the thrombectomy system is configured to selectively deactivate the clot removal state after the clot detection system indicates that a clot is no longer proximate to the distal end of the aspiration catheter.

Clause 11. A thrombectomy system, comprising: an aspiration catheter configured for advancement through vasculature of a subject to facilitate clot removal from the vasculature of the subject; and a clot detection probe extending distally from a distal end of the aspiration catheter, wherein the clot detection probe is configured to emit a signal and to detect a reflected signal that is reflected off of a vessel lumen of the vasculature of the subject to indicate whether a propagation path between the clot detection probe and the vessel lumen is occluded, wherein occlusion of the propagation path between the clot detection probe and the vessel lumen indicates a presence of a clot proximate to the clot detection probe.

Clause 12. The thrombectomy system of clause 11, wherein the clot detection probe extends distally from a distal opening of the aspiration catheter.

Clause 13. The thrombectomy system of clause 12, further comprising one or more fiber optics extending proximally from the clot detection probe into the aspiration catheter.

Clause 14. The thrombectomy system of clause 11, wherein the clot detection probe is configured to emit the signal radially.

Clause 15. The thrombectomy system of clause 11, wherein the signal comprises a light signal.

Clause 16. The thrombectomy system of clause 15, wherein the light signal comprises an optical coherence tomography (OCT) signal.

Clause 17. The thrombectomy system of clause 15, wherein the light signal comprises a light detection and ranging (LiDAR) signal.

Clause 18. The thrombectomy system of clause 11, wherein the signal comprises a sound signal.

Clause 19. The thrombectomy system of clause 11, wherein the aspiration catheter comprises a jet configured to release a propagation medium toward the clot detection probe to enable the signal to propagate through the propagation medium when the propagation path is un-occluded.

Clause 20. The thrombectomy system of clause 11, wherein the thrombectomy system is configured to selectively activate a clot removal state after the clot detection probe indicates that a clot is proximate to the distal end of the aspiration catheter.

Clause 21. The thrombectomy system of clause 20, wherein, during operation of the clot removal state, the thrombectomy system is configured to selectively deactivate the clot removal state after the clot detection probe indicates that a clot is no longer proximate to the distal end of the aspiration catheter.

Clause 22. A thrombectomy system, comprising: an aspiration catheter configured for advancement through vasculature of a subject to facilitate clot removal from the vasculature of the subject; and a clot detection probe positioned proximate to a distal end of the aspiration catheter, wherein the clot detection probe is configured to detect force or pressure exerted on the clot detection probe, and wherein a detected force or pressure exerted on the clot detection probe that satisfies one or more conditions indicates a presence of a clot proximate to the clot detection probe.

Clause 23. The thrombectomy system of clause 22, wherein the clot detection probe is positioned on an inner wall of a distal opening of the aspiration catheter.

Clause 24. The thrombectomy system of clause 22, wherein the clot detection probe comprises a fiber optic pressure or force sensor.

Clause 25. The thrombectomy system of clause 24, further comprising one or more fiber optics extending proximally from the clot detection probe into the aspiration catheter.

Clause 26. The thrombectomy system of clause 22, wherein the thrombectomy system is configured to selectively activate a clot removal state after the clot detection probe indicates that a clot is proximate to the distal end of the aspiration catheter.

Clause 27. The thrombectomy system of clause 26, wherein, during operation of the clot removal state, the thrombectomy system is configured to selectively deactivate the clot removal state after the clot detection probe indicates that a clot is no longer proximate to the distal end of the aspiration catheter.

Clause 28. A thrombectomy system, comprising: an aspiration catheter configured for advancement through vasculature of a subject to facilitate clot removal from the vasculature of the subject; and a clot detection system comprising an anode and a cathode, wherein the anode and the cathode are positioned proximate to a distal end of the aspiration catheter, wherein the clot detection system is configured to apply a fixed voltage current at the anode and the cathode, wherein the clot detection system is configured to measure current and/or resistance during application of the fixed voltage current, and wherein the measured current and/or resistance is indicative of a presence of a clot proximate to the distal end of the aspiration catheter.

Clause 29. The thrombectomy system of clause 28, wherein the anode and the cathode are positioned on an inner wall of a distal opening of the aspiration catheter.

Clause 30. The thrombectomy system of clause 29, wherein the anode and the cathode are offset from one another on the inner wall of the distal opening by about 180°.

Clause 31. The thrombectomy system of clause 28, wherein the thrombectomy system is configured to selectively activate a clot removal state after the clot detection system indicates that a clot is proximate to the distal end of the aspiration catheter.

Clause 32. The thrombectomy system of clause 31, wherein, during operation of the clot removal state, the thrombectomy system is configured to selectively deactivate the clot removal state after the clot detection system indicates that a clot is no longer proximate to the distal end of the aspiration catheter.

### Additional Details Related to Implementing the Disclosed Embodiments

The principles disclosed herein may be implemented in various formats. For example, at least some techniques discussed herein may be performed as a method that includes various acts for achieving particular results or benefits. In some instances, the techniques discussed herein are represented in computer-executable instructions that may be stored on one or more hardware storage devices. The computer-executable instructions may be executable by one or more processors to carry out (or to configure a system to carry out) the disclosed techniques. In some embodiments, a system may be configured to send the computer-executable instructions to a remote device to configure the remote device for carrying out the disclosed techniques.

Systems for implementing the disclosed embodiments may include various components, such as, by way of non-limiting example, processor(s), storage, sensor(s), I/O system(s), communication system(s), etc.

The processor(s) may comprise one or more sets of electronic circuitries that include any number of logic units, registers, and/or control units to facilitate the execution of computer-readable instructions (e.g., instructions that form a computer program). Such computer-readable instructions may be stored within storage. The storage may comprise physical system memory and may be volatile, non-volatile, or some combination thereof. Furthermore, storage may comprise local storage, remote storage (e.g., accessible via communication system(s) or otherwise), or some combination thereof.

In some implementations, the processor(s) may comprise or be configurable to execute any combination of software and/or hardware components that are operable to facilitate processing using machine learning models or other artificial intelligence-based structures/architectures. Artificial intelligence-based structures/architectures may take on any suitable form, such as by comprising or utilizing hardware components and/or computer-executable instructions operable to carry out function blocks and/or processing layers configured in the form of, by way of non-limiting example, single-layer neural networks, feed forward neural networks, radial basis function networks, deep feed-forward networks, recurrent neural networks, long-short term memory (LSTM) networks, gated recurrent units, autoencoder neural networks, variational autoencoders, denoising autoencoders, sparse autoencoders, Markov chains, Hopfield neural networks, Boltzmann machine networks, restricted Boltzmann machine networks, deep belief networks, deep convolutional networks (or convolutional neural networks), deconvolutional neural networks, deep convolutional inverse graphics networks, generative adversarial networks, liquid state machines, extreme learning machines, echo state networks, deep residual networks, Kohonen networks, support vector machines, neural Turing machines, and/or others.

In some instances, actions performable by a system may rely at least in part on communication system(s) for receiving information from remote system(s), which may include, for example, separate systems or computing devices, sensors, and/or others. The communications system(s) may comprise any combination of software or hardware components that are operable to facilitate communication between on-system components/devices and/or with off-system components/devices. For example, the communications system(s) may comprise ports, buses, or other physical connection apparatuses for communicating with other devices/components. Additionally, or alternatively, the communications system(s) may comprise systems/components operable to communicate wirelessly with external systems and/or devices through any suitable communication channel(s), such as, by way of non-limiting example, Bluetooth, ultra-wideband, WLAN, infrared communication, and/or others.

A system may comprise or be in communication with sensor(s). Sensor(s) may comprise any device for capturing or measuring data representative of perceivable phenomenon. By way of non-limiting example, the sensor(s) may comprise one or more image sensors, microphones, thermometers, barometers, magnetometers, accelerometers, gyroscopes, and/or others.

Furthermore, a system may comprise or be in communication with I/O system(s). I/O system(s) may include any type of input or output device such as, by way of non-limiting example, a touch screen, a mouse, a keyboard, a controller, and/or others, without limitation. For example, the I/O system(s) may include a display system that may comprise any number of display panels, optics, laser scanning display assemblies, and/or other components. One will appreciate, in view of the present disclosure, that the sensor(s) may, in some instances, be utilized as I/O system(s).

Disclosed embodiments may comprise or utilize a special purpose or general-purpose computer including computer hardware, as discussed in greater detail below. Disclosed embodiments also include physical and other computer-readable media for carrying or storing computer-executable instructions and/or data structures. Such computer-readable media can be any available media that can be accessed by a general-purpose or special-purpose computer system. Computer-readable media that store computer-executable instructions in the form of data are one or more “computer-readable recording media,” “physical computer storage media,” or “hardware storage device(s).” Computer-readable media that merely carry computer-executable instructions without storing the computer-executable instructions are “transmission media.” Thus, by way of example and not limitation, the current embodiments can comprise at least two distinctly different kinds of computer-readable media: computer storage media and transmission media.

Computer storage media (aka “hardware storage device”) are computer-readable hardware storage devices, such as RAM, ROM, EEPROM, CD-ROM, solid state drives (“SSD”) that are based on RAM, Flash memory, phase-change memory (“PCM”), or other types of memory, or other optical disk storage, magnetic disk storage or other magnetic storage devices, or any other medium that can be used to store desired program code means in hardware in the form of computer-executable instructions, data, or data structures and that can be accessed by a general-purpose or special-purpose computer.

A “network” may comprise one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmission media can include a network and/or data links which can be used to carry program code in the form of computer-executable instructions or data structures and which can be accessed by a general purpose or special purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general-purpose computer, special purpose computer, or special purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Disclosed embodiments may comprise or utilize cloud computing. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, wearable devices, and the like. The invention may also be practiced in distributed system environments where multiple computer systems (e.g., local and remote systems), which are linked through a network (either by hardwired data links, wireless data links, or by a combination of hardwired and wireless data links), perform tasks. In a distributed system environment, program modules may be located in local and/or remote memory storage devices.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Application-specific Standard Products (ASSPs), System-on-a-chip systems (SOCs), Complex Programmable Logic Devices (CPLDs), central processing units (CPUs), graphics processing units (GPUs), and/or others.

As used herein, the terms “executable module,” “executable component,” “component,” “module,” or “engine” can refer to hardware processing units or to software objects, routines, or methods that may be executed on one or more computer systems. The different components, modules, engines, and services described herein may be implemented as objects or processors that execute on one or more computer systems (e.g., as separate threads).

It is contemplated that various combinations or sub-combinations of the specific features and aspects of the embodiments disclosed above may be made and still fall within one or more of the embodiments. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed embodiments. Thus, it is intended that the scope of the present disclosure herein disclosed should not be limited by the particular disclosed embodiments described above. Moreover, while the present disclosure is susceptible to various modifications, and alternative forms, specific examples thereof have been shown in the drawings and are herein described in detail. It should be understood, however, that the present disclosure is not to be limited to the particular forms or methods disclosed, but to the contrary, the present disclosure is to cover all modifications, equivalents, and alternatives falling within the spirit and scope of the various embodiments described and the appended claims.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

For purposes of the present disclosure and appended claims, the conjunction “or” is to be construed inclusively (e.g., “an apple or an orange” would be interpreted as “an apple, or an orange, or both”; e.g., “an apple, an orange, or an avocado” would be interpreted as “an apple, or an orange, or an avocado, or any two, or all three”), unless: (i) it is explicitly stated otherwise, e.g., by use of “either . . . or,” “only one of,” or similar language; or (ii) two or more of the listed alternatives are mutually exclusive within the particular context, in which case “or” would encompass only those combinations involving non-mutually-exclusive alternatives. For purposes of the present disclosure and appended claims, the words “comprising,” “including,” “having,” and variants thereof, wherever they appear, shall be construed as open-ended terminology, with the same meaning as if the phrase “at least” were appended after each instance thereof.

We claim:

1. A thrombectomy system, comprising:
an aspiration catheter configured for advancement through vasculature of a subject to facilitate clot removal from the vasculature of the subject; and
a clot detection system comprising an emitter and a detector, wherein the emitter and the detector are positioned proximate to a distal end of the aspiration catheter, wherein the detector is configured to detect emissions of the emitter to indicate whether a propagation path between the emitter and the detector is occluded, wherein occlusion of the propagation path between the emitter and the detector indicates a presence of a clot proximate to the distal end of the aspiration catheter.

2. The thrombectomy system of claim 1, wherein the emitter and the detector are positioned on an inner wall of a distal opening of the aspiration catheter.

3. The thrombectomy system of claim 2, wherein the clot detection system further comprises one or more fiber optics extending proximally from the emitter and the detector within the aspiration catheter.

4. The thrombectomy system of claim 1, wherein the emissions of the emitter comprise light emissions or sound emissions.

5. The thrombectomy system of claim 1, wherein the aspiration catheter comprises a jet configured to release a propagation medium toward the propagation path to enable the emissions of the emitter to propagate through the propagation medium when the propagation path is un-occluded.

6. The thrombectomy system of claim 1, wherein the thrombectomy system is configured to selectively activate a clot removal state after the clot detection system indicates that a clot is proximate to the distal end of the aspiration catheter.

7. A thrombectomy system, comprising:
of a subject to facilitate clot removal from the vasculature of the subject; and
a clot detection probe extending distally from a distal end of the aspiration catheter, wherein the clot detection probe is configured to emit a signal and to detect a reflected signal that is reflected off of a vessel lumen of the vasculature of the subject to indicate whether a propagation path between the clot detection probe and the vessel lumen is occluded, wherein occlusion of the propagation path between the clot detection probe and the vessel lumen indicates a presence of a clot proximate to the clot detection probe.

8. The thrombectomy system of claim 7, wherein the clot detection probe extends distally from a distal opening of the aspiration catheter.

9. The thrombectomy system of claim 8, further comprising one or more fiber optics extending proximally from the clot detection probe into the aspiration catheter.

10. The thrombectomy system of claim 7, wherein the signal comprises a light signal or a sound signal.

11. The thrombectomy system of claim 10, wherein the light signal comprises an optical coherence tomography (OCT) signal or a light detection and ranging (LiDAR) signal.

12. The thrombectomy system of claim 7, wherein the aspiration catheter comprises a jet configured to release a propagation medium toward the clot detection probe to enable the signal to propagate through the propagation medium when the propagation path is un-occluded.

13. The thrombectomy system of claim 7, wherein the thrombectomy system is configured to selectively activate a clot removal state after the clot detection probe indicates that a clot is proximate to the distal end of the aspiration catheter.

14. The thrombectomy system of claim 13, wherein, during operation of the clot removal state, the thrombectomy system is configured to selectively deactivate the clot removal state in response to the clot detection probe indicating that a clot is no longer proximate to the distal end of the aspiration catheter.

15. A thrombectomy system, comprising:
of a subject to facilitate clot removal from the vasculature of the subject; and
a clot detection probe positioned proximate to a distal end of the aspiration catheter, wherein the clot detection probe is configured to detect force or pressure exerted on the clot detection probe, and wherein a detected force or pressure exerted on the clot detection probe that satisfies one or more conditions indicates a presence of a clot proximate to the clot detection probe.

16. The thrombectomy system of claim 15, wherein the clot detection probe comprises a fiber optic pressure or force sensor.

17. The thrombectomy system of claim 15, wherein the thrombectomy system is configured to selectively activate a clot removal state after the clot detection probe indicates that a clot is proximate to the distal end of the aspiration catheter.

18. A thrombectomy system, comprising:
of a subject to facilitate clot removal from the vasculature of the subject; and
a clot detection system comprising an anode and a cathode, wherein the anode and the cathode are positioned proximate to a distal end of the aspiration catheter, wherein the clot detection system is configured to apply a fixed voltage current at the anode and the cathode, wherein the clot detection system is configured to measure current and/or resistance during application of the fixed voltage current, and wherein the measured current and/or resistance is indicative of a presence of a clot proximate to the distal end of the aspiration catheter.

19. The thrombectomy system of claim 18, wherein the anode and the cathode are positioned on an inner wall of a distal opening of the aspiration catheter.

20. The thrombectomy system of claim 18, wherein the thrombectomy system is configured to selectively activate a clot removal state after the clot detection system indicates that a clot is proximate to the distal end of the aspiration catheter.
